(19) United States
(12) Patent Application Publication
(10) Pub. No.: US 2008/0103352 A1
(43) Pub. Date: May 1, 2008

(54) Implantable Medical Devices Having Recesses

(76) Inventors: Nicolas C. Rivron, Juvisy sur Orge (FR); Paul V. Trescony, Champlin, MN (US); Michael F. Wolf, Golden Valley, MN (US)

Correspondence Address: HAUGEN LAW FIRM, SUITE 1130 - TCF TOWER, 121 SOUTH EIGHTH STREET, MINNEAPOLIS, MN, 55402, US

(21) Appl. No.: 11/971,534

(22) Filed: Jan. 9, 2008

Related U.S. Application Data
(63) Continuation of application No. 10/656,855, filed on Sep. 4, 2003.

(51) Int. Cl. A61F 2/06

(52) U.S. Cl. 600/36; 623/1.39

(57) ABSTRACT

In general, the invention is directed to devices and methods that are useful for surface preparation of implantable medical devices. In the case of a vascular graft, the invention presents devices and methods that enhance endothelial cell seeding by providing recesses in the luminal surface that can receive endothelial cells. When the device is constructed of a material such as expanded polytetrafluoroethylene (ePTFE), the recesses may be created by physical processing of the microstructures of the material. The physical processing lifts nodes from the surface, forming recesses that can receive endothelial cells.

### CROSS-REFERENCE TO RELATED APPLICATION

The present application is a continuation of co-pending application Ser. No. 10/656,855, filed Sep. 4, 2003, entitled “Implantable Medical Devices Having Recesses”, the content of which is incorporated herein in its entirety.

### FIELD OF THE INVENTION

The invention relates to materials and devices implantable in a human body, such as materials and devices used in vascular prostheses.

### BACKGROUND OF THE INVENTION

Some patients develop conditions that can be corrected with surgical grafts. In particular, conditions that affect blood flow through the vessels of the body may be treated with vascular grafts, in which a surgeon applies the graft to supplant the damaged vascular tissue. Coronary artery disease, peripheral vascular disease and end stage renal disease are examples of conditions in which vascular flow is affected, and which can be addressed with surgical grafts.

Vascular grafts may be autologous, i.e., the graft may be taken from the patient for transplantation at another site. In some cases, however, an autologous graft may not be feasible, and a synthetic vascular graft may be employed instead. A synthetic vascular graft is a tube-shaped prosthesis made of a biocompatible material such as expanded polytetrafluoroethylene (ePTFE). The synthetic vascular graft includes a lumen through which blood flows.

In a vessel, the intima is the layer closest to the lumen where blood flows. It is made up mainly of a monolayer of endothelial cells attached to a basement membrane and matrix molecules. The endothelial cells are specialized cells that line the lumen of blood vessels, and play several roles. Endothelial cells secrete vasoactive substances, for example, and secrete substances that stimulate new vessel growth and promote or inhibit proliferation of smooth muscle cells in vessel walls in response to hemodynamic demands. Endothelial cells are also influential in formation and dissolution of thrombus, which is a precipitate of blood components that can restrict blood flow through the vessel lumen.

In humans, implanted vascular grafts typically heal by formation of an acellular psuedo-intima without large-scale outgrowth of the native endothelial cell lining. It has been discovered that it is highly beneficial for a synthetic vascular graft to include a layer of endothelial cells in the lumen, to prevent thrombosis and to suppress abnormal smooth muscle cell proliferation that could lead to stenosis or narrowing of the vessel. To promote the formation of a homogeneous, dense and confluent layer of endothelial cells inside the synthetic vascular graft, techniques have been developed for “endothelial cell seeding” of vascular grafts. In general, this “seeding” or deposition of cells involves harvesting autologous endothelial cells and transplanting the harvested cells to the lumen of the synthetic vascular graft.

### SUMMARY OF THE INVENTION

In general, the invention is directed to devices and methods that are useful for surface preparation of implantable medical devices. In the case of a vascular graft, the invention presents devices and methods that enhance endothelial cell seeding. The invention includes a vascular prosthesis that includes recesses in the luminal surface that can receive endothelial cells. The recesses are oriented at least partially along the luminal direction, and represent “grooves,” “wells,” “harbors,” “pockets” or “hiding spaces” for the endothelial cells.

When the implantable device is constructed of a material such as expanded polytetrafluoroethylene (ePTFE), the recesses may be created by physical processing of the microstructures of the material. In a vascular prosthesis made of ePTFE, the luminal surface of the prosthesis includes microscopic nodes and fibrils (or fibers) that cooperate to give the material its strength and physical properties. By physically processing the luminal surface, such as by rubbing or applying force to the surface with a pressurized fluid, nodes can be lifted from the luminal surface, forming recesses that can receive the endothelial cells.

In the absence of recesses, endothelial cells deposited on the lumen of a synthetic vascular graft tend to be exposed and washed away by the flow of blood. Even when the cells adhere to the luminal surface, the shear forces associated with fluid flow often overcome the adhesion and wash the endothelial cells away. When the endothelial cells are washed away, the vessel is less likely to endothelialize and is at greater risk of developing complications, such as thrombosis and stenosis.

The shear forces wash away fewer endothelial cells, however, when the endothelial cells reside in recesses according to the invention. The fluid flow is less likely to dislodge and wash away endothelial cells in the recesses. With time, the endothelial cells grow in situ under physiological conditions, mature and colonize the graft lumen.

In one embodiment, the invention is directed to a device comprising a vascular prosthesis. The prosthesis includes a luminal surface that defines a luminal direction. The luminal surface comprises a plurality of recesses sized to receive at least one endothelial cell, and the recesses are oriented at least partially along the luminal direction. The vascular prosthesis may be made of ePTFE or another material.

In another embodiment, the invention is directed to a medical device adapted to be implanted in a human body. The medical device includes at least one surface that includes ePTFE. The surface comprises nodes formed of polytetrafluoroethylene (PTFE), and the surface includes recesses defined by nodes lifted from the surface. This embodiment of the invention may be realized as a vascular prosthesis or as another medical device.

In a further embodiment, the invention is directed to a method comprising rubbing a luminal surface of a vascular prosthesis with a tool. The tool may be, for example, a wheel brush with bristles of metal or nylon.

In an additional embodiment, the invention presents a method comprising applying a force to a medical device. The medical device is adapted to be implanted in a human body and includes at least one surface including ePTFE. The application of force lifts nodes from the surface to define a plurality of recesses. The force may be applied by, for example, rubbing the surface with a tool or by applying a pressurized fluid to the surface.

In an added embodiment, the invention is directed to a method comprising seeding endothelial cells on a medical device adapted to be implanted in a human body. The medical device includes at least one surface that includes ePTFE, and this surface comprises nodes formed of PTFE, and the surface includes recesses defined by nodes lifted from the surface.

The invention may result in one or more advantages. In the case of a vascular prosthesis manufactured according to the invention, fewer endothelial cells will be washed away when the prosthesis is implanted, thereby benefiting the patient. Also, various embodiments of the invention take advantage of physical properties of ePTFE, a material that has a proven track record in implantable medical devices. The invention improves ePTFE without adversely affecting the favorable features of ePTFE, such as biocompatibility, and ease of handling and suturing.

In addition, the invention also makes a “one-stage procedure” feasible, in which endothelial cells can be harvested, a prosthesis can be seeded with the harvested cells, and the seeded prosthesis can be presented for implantation in a single surgical operation.

The details of one or more embodiments of the invention are set forth in the accompanying drawings and the description below. Other features, objects, and advantages of the invention will be apparent from the description and drawings, and from the claims.

### BRIEF DESCRIPTION OF THE DRAWINGS

FIG. 1 is a perspective view of a vascular prosthesis.

FIG. 2 is a perspective view of a tool assembly for processing a vascular prosthesis.

FIG. 3 is a scanning electron microscope (SEM) image of expanded polytetrafluoroethylene (ePTFE) material prior to processing according to the invention.

FIG. 4 is an SEM image of ePTFE material after processing according to the invention.

FIG. 5 is an SEM image of ePTFE material after processing according to the invention, shown in cross-section and at an oblique angle.

FIG. 6 is an SEM image of ePTFE material after processing according to the invention, seeded with endothelial cells.

FIG. 7 is a diagram illustrating the structure of ePTFE material.

FIGS. 8-10 are diagrams illustrating exemplary techniques for rubbing ePTFE material with a tool.

FIG. 11 is a flow diagram illustrating a technique for processing a vascular prosthesis according to the invention.

FIG. 12 is a flow diagram illustrating an implantation technique according to the invention.

### DETAILED DESCRIPTION OF THE PREFERRED EMBODIMENTS

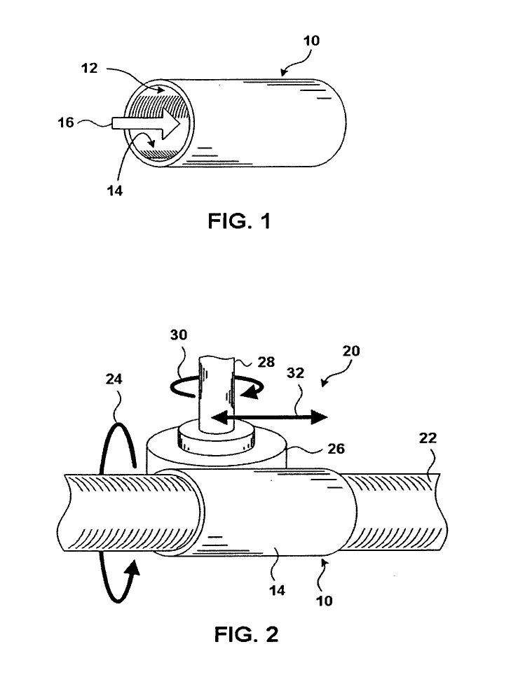

FIG. 1 is a diagram of a vascular prosthesis 10 according to the invention. Prosthesis 10 is a generally tube-shaped structure that includes a lumen 12 through which a fluid can flow. In a typical application, vascular prosthesis 10 supplants a blood vessel, and the fluid that flows through lumen 12 is blood. A luminal surface 14 of vascular prosthesis 10 comes in contact with the blood.

The geometry of luminal surface 14 of vascular prosthesis 10 defines a “luminal direction,” which is along the axis of the tubular prosthesis. Although fluid may physically flow through lumen 12 forward or backward along the luminal direction, fluid generally flows predominantly in one direction in an implanted environment. It is therefore useful to define a “flow direction” which represents a particular direction of fluid flow. In FIGS. 1 and 3-5, arrow 16 identifies the flow direction. Flow direction 16 is coincident with the luminal direction, but is directed in a single direction. Fluid moving in flow direction 16 may be considered as moving “forward,” and fluid moving opposite flow direction 16 may be considered as moving “backward.”

FIG. 2 is a diagram of an exemplary tool assembly 20 that processes vascular prosthesis 10 by rubbing vascular prosthesis 10. “Rubbing” comprises any process that includes moving a tool with pressure relative to vascular prosthesis 10, such as by scraping, scoring, abrading, brushing, chafing, scratching or scuffing.

As shown in FIG. 2, vascular prosthesis 10 has been everted, i.e., vascular prosthesis 10 has been turned “inside out” to facilitate processing with tool assembly 20. Vascular prosthesis 10 has been mounted on a rotatable supporting mandrel 22, which may be free to rotate as shown by directional arrow 24.

A tool 26 rubs luminal surface 14. In exemplary tool assembly 20, tool 26 is mounted on a rotating shaft 28 that rotates as shown by directional arrow 30. When tool 26 is brought in contact with luminal surface 14 and rotated, tool 26 rubs against luminal surface 14. Mandrel 22 or shaft 28 or both further have freedom to move in a transverse direction, as shown by directional arrow 32.

By rotating tool 26 and moving tool and prosthesis 10 transversely to one another, and by rotating mandrel 22, tool 26 can be brought into contact with any point on luminal surface 14. In this way, tool 26 can rub the entire luminal surface 14. Although not essential for the invention, there are advantages to rubbing the entire luminal surface, as will be described below.

When vascular prosthesis is constructed of a material such as expanded polytetrafluoroethylene (ePTFE), rubbing luminal surface 14 with tool 26 creates recesses in the microstructures of luminal surface 14. In particular, rubbing luminal surface 14 lifts microscopic “nodes” from luminal surface 14, forming recesses that can receive seeded autologous endothelial cells. As used herein, “endothelial cells” includes endothelial precursor or stem cells, as well as developed endothelial cells.

Tool 26 may be any of several tools. Tool 26 may be solid, such as a rotating drum of metal, plastic, rubber or ceramic. Tool 26 may also include a wheel brush with bristles. The bristles may be constructed of any material, including metal, plastic, rubber or ceramic. Through experimentation, it has been discovered that a wheel brush with metal bristles, such as brass or stainless steel bristles, can generate recesses in the luminal surface. A wheel brush with nylon bristles also is effective in generating recesses. A technique for rubbing a luminal surface of a vascular prosthesis with a tool will be described below.

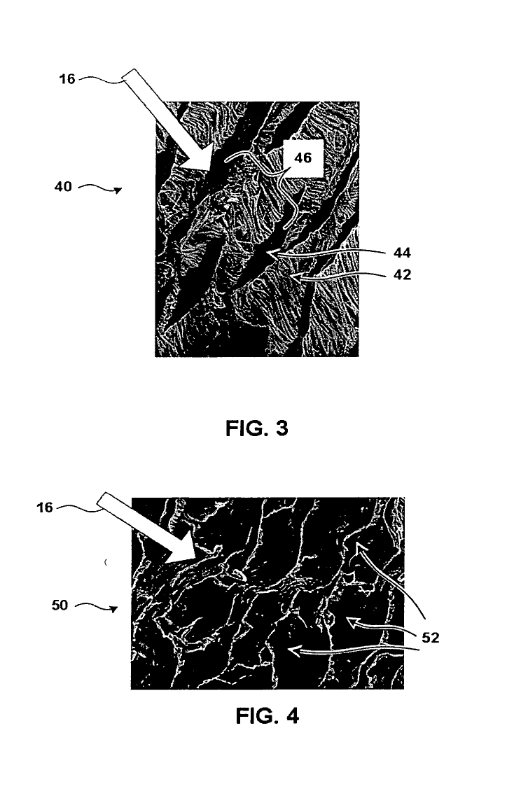

FIG. 3 is an image of ePTFE material 40 taken by a scanning electron microscope (SEM). The image of FIG. 3 depicts ePTFE material 40 such as that found in a standard vascular graft such as that shown in FIG. 1. In particular, the image of FIG. 3 depicts a microscopic view of the luminal surface of a prosthesis, i.e., the surface that may be in contact with a flowing bodily fluid, such as blood.

Two types of microstructures provide ePTFE material 40 with its strength and other physical properties, and these microstructures are evident on the luminal surface shown in FIG. 3. In particular, ePTFE material 40 includes thin polytetrafluoroethylene (PTFE) fibrils 42 draped between the much thicker islands or “nodes” 44 of PTFE. The orientations of fibrils 42 and nodes 44 are substantially perpendicular to one another, and result from the manufacture of ePTFE.

In general, the manufacture of ePTFE includes preparation of a material that includes PTFE particles that have been fused together. At one stage in the manufacturing process, the material is stretched or “expanded.” The expansion causes fibrils 42 to form in the direction of the expansion, giving ePTFE directionality. The degree of expansion also affects the internodal distance, i.e., the average distance between neighboring nodes in the direction of expansion. Internodal distances may be, for example on the order of about 30 to 90 micrometers. Reference numeral 46 shows a typical internodal distance.

In FIG. 3, ePTFE material 40 has not yet been rubbed with a tool. For reference, FIG. 3 shows flow direction 16. Flow direction 16 is substantially perpendicular to the orientation of nodes 44, and substantially parallel to the orientation of fibrils 42.

FIG. 4 is an image of ePTFE material 40 taken by an SEM. Material 40 has been subjected to preparation, thereby creating a plurality of recesses 52 in the luminal surface. As will be described below, rubbing the luminal surface with a tool generates recesses 52. Recesses 52 can receive endothelial cells. Recesses 52 represent “grooves,” “wells,” “harbors,” “pockets” or “hiding spaces” for the endothelial cells.

As shown in FIG. 4, recesses 52 are oriented at least partially along the luminal direction. In particular, the recesses extend into the luminal surface, but extend at least partially in the direction opposite flow direction 16. In other words, a fluid moving in flow direction 16 would generally flow over recesses 52, rather than into recesses 52.

As shown in FIG. 4, the luminal surface affects the fibrils network visible in FIG. 3. As a result of rubbing, many of the fibrils are disrupted, resulting in smooth, fibril-free surfaces. This effect is generally restricted to the luminal surface, however. Fibrils beneath the luminal surface are largely intact, imparting strength and other physical properties to material 50. In addition, fibrils may reside inside recesses 52. It has been discovered through experimentation that the extent of smooth, fibril-free surfaces is generally a function of the extent of rubbing.

Viewed with an SEM, the luminal surface of material 50 resembles a series of overlapping layers. The layers separate from one another in a scale-like texture that resembles a “fish-scale” pattern, creating recesses that can harbor endothelial cells.

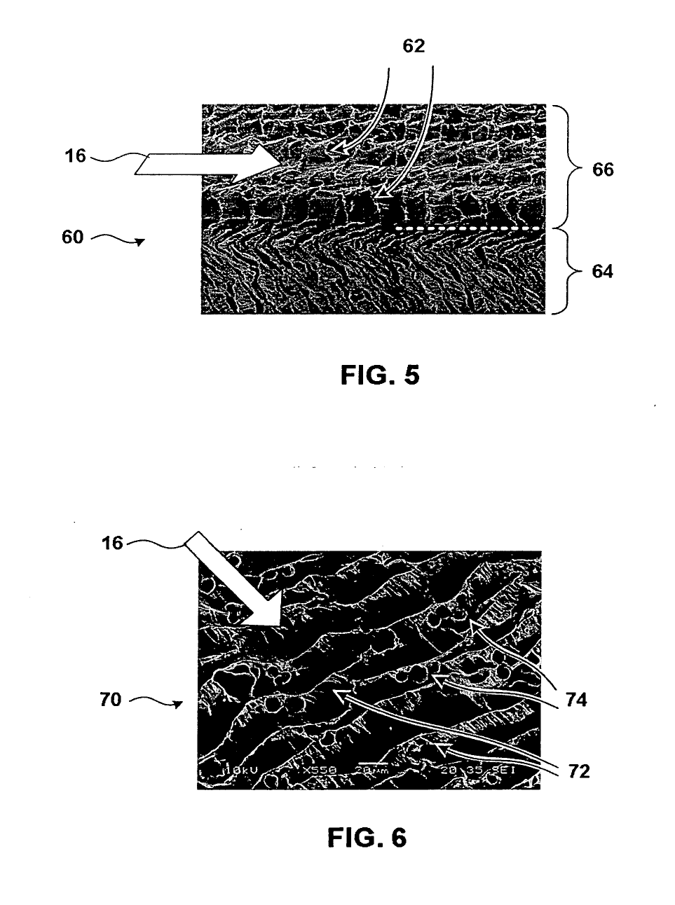

FIG. 5 is an image of ePTFE material 60 taken by an SEM that shows the structure of material 60 following preparation and creation of recesses 62. FIG. 5 shows in part a cross section 64 of material 60, i.e., material beneath the luminal surface. Although rubbing has affected the luminal surface, the material below the luminal surface maintains its structure. In a typical vascular prosthesis having a wall thickness of three-tenths to five-tenths of a millimeter, rubbing would generally affect no more than five to ten percent of the thickness of the material.

FIG. 5 also provides an oblique view 66 of the luminal surface. As can be seen from oblique view 66, recesses 62 are oriented at least partially along the luminal direction, and extend into the luminal surface at least partially in the direction opposite flow direction 16.

FIG. 6 is an image of ePTFE material 70 taken by an SEM. Material 70 is similar to material 50 in FIG. 4, and material 60 in FIG. 5, but material 70 includes recesses 72 in the luminal surface and endothelial cells 74 received in recesses 72. As shown in FIG. 6, a fluid moving in flow direction 16 would generally flow over recesses 72 and over cells 74. As a result, a cell residing in a recess is subjected to less shear force from the fluid than a cell outside a recess, and is less likely to be exposed and washed away by the fluid.

In a conventional vascular prosthesis seeded with endothelial cells, the endothelial cells deposited on the lumen of the prosthesis tend to be washed away by the flow of blood. Even when the cells adhere to the luminal surface, the shear forces associated with fluid flow often overcome the adhesion and wash the endothelial cells away. When the endothelial cells are washed away, the vessel is less likely to endothelialize and is at greater risk of developing complications, such as thrombosis and stenosis.

In a vascular prosthesis with a luminal surface such as shown in FIG. 6, however, shear forces may wash away fewer endothelial cells. Because endothelial cells 74 reside in recesses 72, fluid flow along fluid direction 16 is less likely to dislodge and wash away endothelial cells 74 in recesses 72. With time, endothelial cells 74 grow in situ, mature and colonize the luminal surface, with recesses 72 providing a foundation for growth and colonization. As result, the vascular prosthesis maintains a population of endothelial cells that help reduce the risk of complications.

In addition, rubbing results in smooth, fibril-free surfaces. Endothelial cells 74 typically adhere more efficiently to smooth nodal surfaces than to fibrils. Rubbing the luminal surface with a tool, in addition to creating recesses, also creates a more suitable surface for cell adhesion.

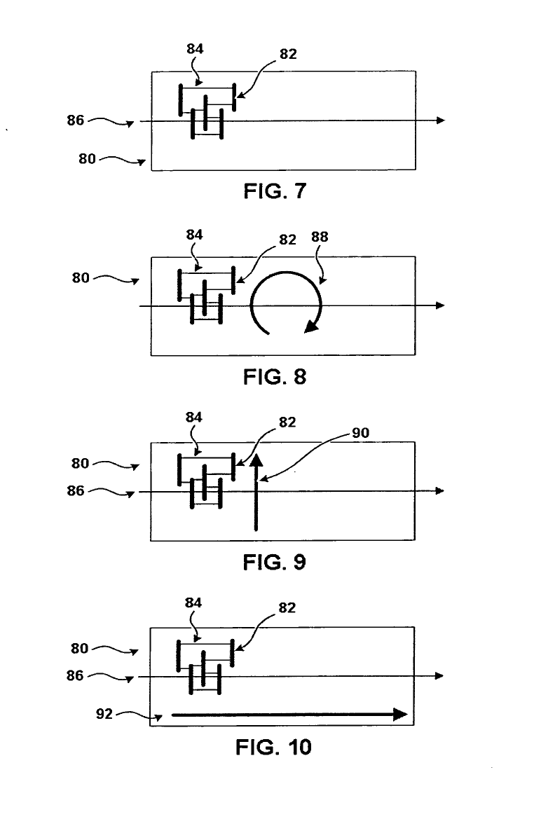

As noted above, the manufacture of ePTFE includes an expansion that imparts directionality to ePTFE. FIG. 7 is a diagram of an ePTFE sample 80 that illustrates the directionality of ePTFE material. In FIG. 7, sample 80 includes nodes 82 and fibrils 84. Arrow 86 identifies a direction that is substantially perpendicular to the orientation of nodes 82, and substantially parallel to the orientation of fibrils 84. FIGS. 8-10 are diagrams illustrating techniques for rubbing ePTFE sample 80 with a tool.

As shown in FIG. 8, one technique for rubbing sample 80 includes rotational rubbing with a tool such as a wheel brush. Rotational rubbing may be accomplished using tool assembly 20 shown in FIG. 2 by bringing the circular face of tool 26, rather than the side of tool 26, into contact with prosthesis 10. With rotational rubbing, the tool rubs the luminal surface in many directions 88 simultaneously. Some of the rubbing may be substantially parallel to the orientation of nodes 82, and some may be substantially perpendicular to the orientation of nodes 82.

FIG. 9, illustrates another technique for rubbing, i.e., radial rubbing with a tool. Radial rubbing comprises rubbing the luminal surface of sample 80 in a direction 90 that is substantially parallel to the orientation of nodes 82, and substantially perpendicular to the orientation of fibrils 84. Rotational rubbing may be accomplished using tool assembly 20 shown in FIG. 2 by bringing the side of tool 26 into contact with prosthesis 10, and orienting mandrel 22 and shaft 28 in the same direction.

A further technique, shown in FIG. 10, includes transverse rubbing of sample 80 with a tool. Transverse rubbing comprises rubbing the luminal surface in a direction 92 that is substantially perpendicular to the orientation of nodes 82, and substantially parallel to the orientation of fibrils 84. FIG. 2 depicts tool assembly 20 rubbing vascular prosthesis 10 in a transverse direction.

Through experimentation, it has been discovered that transverse rubbing as depicted in FIG. 10, is effective in lifting nodes from the luminal surface to define a plurality of recesses. Radial rubbing, as depicted in FIG. 9, tends to disrupt fibrils 84 without lifting large numbers of nodes 82 to create recesses. Rotational rubbing, as depicted in FIG. 8, tends to produce regions in which nodes are lifted, comparable to the effect of transverse rubbing, and regions in which nodes are not lifted, comparable to the effect of radial rubbing.

It is possible to rub sample 80 with a tool in multiple directions simultaneously. For example, it is possible to rub sample 80 in a direction that has a radial rubbing component and a transverse rubbing component. In general, the greater the transverse rubbing in relation to the radial rubbing, the more nodes are lifted and the more recesses are created. It is also possible to repeat rubbing of the same region of sample 80 in the same way or a different way. Repeat rubbing can further refine the structure of the formed recesses.

Translational rubbing disrupts fibrils 84 on the luminal surface, but also lifts or “plucks” nodes from the luminal surface, thereby creating recesses oriented at least partially along the luminal direction. There may be one or more mechanisms that cause the nodes to be lifted from the luminal surface. When the tool used to rub the luminal surface is a wheel brush with bristles, for example, the bristles may contact nodes and lift the nodes from the luminal surface by friction. The contact between the tool and the surface may also facilitate PTFE “smearing,” in which PTFE structures spreads and merge with one another, generating recesses in the process.

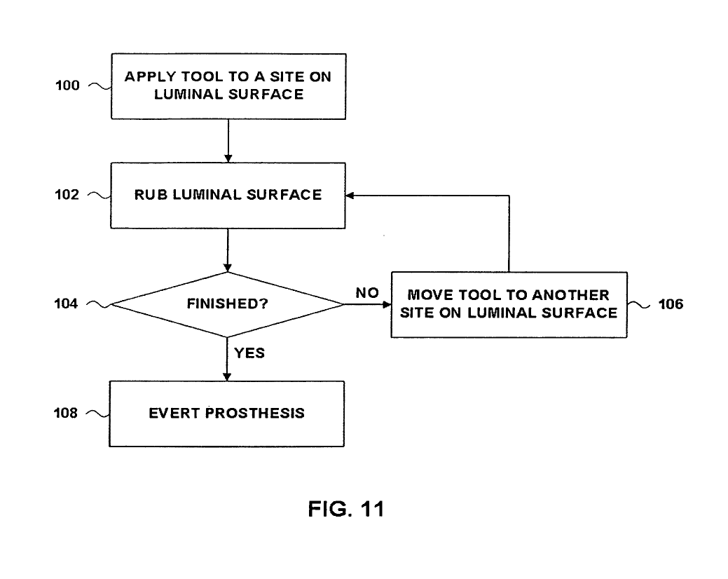

FIG. 11 is a flow diagram illustrating a process for preparing a luminal surface of a vascular prosthesis. The process includes applying a tool to a site on the luminal surface (100) rubbing the luminal surface with the tool (102). The rubbing lifts nodes, thereby creating recesses oriented at least partially along the luminal direction.

Exemplary tool assembly 20 shown in FIG. 2 depicts vascular prosthesis 10 mounted on a rotatable supporting mandrel 22, with tool 26 brought in contact with luminal surface 14 of vascular prosthesis 10. Tool 26 rubs luminal surface 14 of vascular prosthesis 10 when rotating shaft 28 rotates. By rotating tool 26 and moving tool and prosthesis 10 transversely to one another, and by rotating supporting mandrel 22, tool 26 can be brought into contact with any point on luminal surface 14.

Accordingly, once a site on the luminal surface has been rubbed, the process includes determining whether other sites need to be rubbed as well (104). In some circumstance, the entire luminal surface of the prosthesis may be rubbed. In other circumstances, it may be desirable to seed endothelial cells at specified sites, and only these specified sites will be rubbed. These specified sites may form patterns, such as longitudinal or radial patterns. By selection of specific sites for rubbing, it is possible to create “paths” for cell growth in situ.

If additional rubbing is indicated, the tool is applied to another site (106) and the process is continued (102). When tool 26 has completed rubbing, the prosthesis may be everted for implantation (108), if necessary. In some embodiments, and everted prosthesis may be rubbed again, thereby processing the abluminal surface as well as the luminal surface.

It is believed to be possible to rub a luminal surface without everting the prosthesis, e.g., by running a brush through the lumen one or more times. Accordingly, everting the prosthesis for processing is not essential to the invention. Even so, mounting the prosthesis on a supporting mandrel, as shown in FIG. 2, may allow for very precise control of the rubbing.

In one embodiment of the invention, a 4 millimeter diameter ePTFE vascular graft was everted, placed over a mandrel attached to a tooling jig parallel to the rotational axis of a model lathe via an adjustable loading spring, and the tooling jig fixed to the tool stock of an EMCO Unimat PC model lathe. A wheel brush with densely packed nylon bristles (The Mill-Rose Company, Mentor Ohio, Catalog No. 71810, 1 inch (2.5 cm) diameter, 0.006 inch (150 micrometer) in diameter bristles) was secured in the chuck of a vertical milling head attached to the model lathe. The tool stock was positioned to place the everted graft in contact with the brush attached to the vertical milling head. Uniform translation of the graft across the brush was achieved by attaching the tool stock lead screw to either a 2 rpm or a 10 rpm synchronous motor. While the brush was rotated at speeds ranging from 350 to 2500 rpm, the graft was first passed in one direction across the brush at 0.075 inches (1.9 mm) per minute (2 rpm synchronous motor) or 0.375 inches (9.5 mm) per minute (10 rpm synchronous motor) with a contact force of 15 gram weight (0.033 lb). The graft was then passed a second time across the rotating brush in the opposite direction with a contact force of 55 gram weight (0.12 lb) over the same range of brush rotation and tool stock translation speeds. The ePTFE may have a wide range of average internodal distances, e.g., from 10 to 200 micrometers between nodes, but good results were obtained with average internodal distances in the range of 30 to 90 micrometers. Vascular grafts of ePTFE are available from a variety of manufacturers.

In one embodiment of the invention, a wheel brush with densely packed nylon bristles (Mill-rose No. 71810, 1 inch (2.5 cm) in diameter, each bristle about 0.006 inches (150 micrometers) in diameter) was rotated at 350 to 2500 revolutions per minute against a vascular prosthesis made of ePTFE. The prosthesis had been everted so that that luminal surface was more accessible. The brush was moved along the prosthesis transversely at 1100 to 6500 inches per minute (28 to 165 meters per second). Forces in the range of 30 to 100 grams weight (0.066 to 0.22 pounds) were applied between the brush and the luminal surface. The ePTFE may have a wide range of average internodal distances, e.g., from 10 to 200 micrometers between nodes, but good results were obtained with average internodal distances in the range of 30 to 90 micrometers. Vascular grafts of ePTFE are available from a variety of manufacturers.

Brushing as described above does not necessarily lift every node in the surface, nor does it necessarily lift all nodes to the same degree. It is not uncommon, however, for a node to be lifted from the surface by many times its normal height.

The process depicted in FIG. 11 is not necessarily restricted to vascular grafts. Implantable devices other than vascular grafts may include ePTFE, and may benefit from having surface recesses for harboring endothelial or other cells, such as cells that improve healing following implantation. Even if not seeded with cells, the implantable devices may realize benefits from having surfaces undergo a process such as that depicted in FIG. 11. For example, the surfaces may improve healing or decrease fibrous capsule formation. Implantable devices that may include ePTFE, and that may benefit from having surface recesses may include, for example, implantable prostheses for plastic surgery, artificial ligaments, annuloplasty rings, vascular patches, tubes for neural cell growth, sheathed stents, cardiac assist devices, sensors, pacemaker leads, catheters, shunts, sutures and heart valve sewing rings. Such devices may be implantable on a permanently or a temporary basis.

In addition, when the vascular prosthesis or other implantable device is made from ePTFE, the invention is not limited to physical rubbing with a solid tool. It is believed that nodes may be lifted from the surface of ePTFE by application of a pressurized fluid, such as air or water, to a surface made of ePTFE. In other words, an air jet or water jet may supply sufficient friction to lift nodes so as to define a plurality of recesses. Rubbing or application of a pressurized fluid applies a force to the ePTFE, thereby lifting nodes to define recesses. These techniques are not exclusive of one another. For example, a tool may rub the surface of ePTFE when the surface is coated with a liquid.

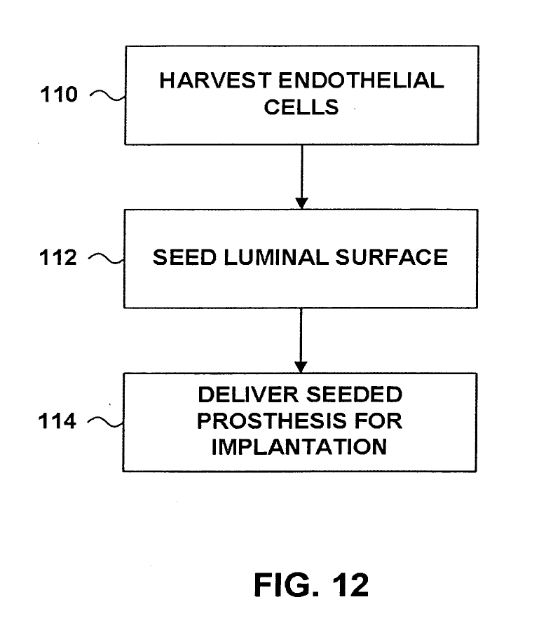

FIG. 12 is a flow diagram showing a technique for preparation of a vascular prosthesis for implantation. FIG. 12 depicts a “one-stage procedure,” i.e., a procedure for preparation of a vascular prosthesis during a single surgical operation.

The technique of FIG. 12 includes harvesting endothelial cells (110). In a typical operation to repair a damaged vessel with a prosthesis, a surgeon retrieves a source of endothelial cells from the patient before or during the procedure to repair the damaged vessel. A surgeon may, for example, retrieve an expendable subdermal vein that includes endothelial cells, and supply the vein to the medical staff for harvesting of the cells. While the staff harvests the cells and prepares the prosthesis, the surgeon may begin repairing the damaged vessel, e.g., obtaining access to the implantation site and preparing the site to receive the prosthesis.

The staff may harvest the cells (110) using any harvesting method. The cells may be separated form the supplied vein and placed in suspension. The staff seeds the prosthesis with harvested endothelial cells (112). The prosthesis is a device having a plurality of recesses sized to receive endothelial cells, with at least some of the recesses oriented at least partially along the luminal direction. The prosthesis will ordinarily have been brought into the operating room with the recesses already formed, and with the prosthesis ready for seeding. The prosthesis may also be premarked to indicate to the surgeon the intended direction of fluid flow through the lumen.

Any seeding method (112) may be used. For example, the fluid with suspended endothelial cells may be introduced into the lumen of the prosthesis, and the prosthesis may be spun with a centrifuge to cause the cells to come in contact with the luminal surface and be received in the recesses. Following seeding, the seeded prosthesis is supplied to the surgeon for implantation (114). Harvesting and seeding in this way can be accomplished quickly, typically in sixty minutes or less, and sometimes in fifteen minutes or less.

This “one-stage procedure” has significant advantages over a conventional “two-stage procedure” for preparation of a vascular prosthesis for implantation. The “two-stage procedure” involves two surgical operations, typically separated by a month or more. In the first operation, the surgeon retrieves a source of endothelial cells from the patient. The surgeon does not implant a prosthesis during this first surgical operation. The medical staff harvests the endothelial cells, and cultures the cells (i.e., grows the cells in vitro) to increase their numbers. Culturing typically takes several weeks. Thereafter, the patient undergoes a second surgical operation to implant a seeded prosthesis. The medical staff seeds the prosthesis, and waits for a period after seeding to allow the cells to adhere to the prosthesis. Seeding may also entail employing adhesion-promoting substances, such as fibrin glue, that promote adhesion. After the waiting period, the medical staff supplies the seeded prosthesis to the surgeon for implantation.

The “one-stage procedure” shown in FIG. 12 has the patient make a single visit to the operating room, rather than two visits, with harvesting and implantation accomplished during this single visit. A single surgical procedure significantly benefits the patient in terms of convenience, comfort and cost.

The “one-stage procedure” omits culturing. In general, the purpose of culturing is to grow enough endothelial cells to compensate for cell losses that occur due to washing away, and to form a confluent monolayer in the lumen. In the one-stage procedure, there is less risk of cells washing away because the seeded cells are received in the luminal surface of the prosthesis.

The one-stage procedure also omits the waiting period that allows the cells to adhere to the prosthesis after seeding. Because the recesses receive the cells, the cells are protected from washing away and can improve adhesion in vivo. Adhesion-promoting substances may be unnecessary. Administration of anticoagulant drugs can control the thrombotic potential of the prosthesis until the seeded prosthesis can form a confluent endothelial cell lining in the lumen. In addition, the one-stage procedure permits cells to grow under physiological conditions of pressure and shear stress, which promotes the formation of a more dense and orientated endothelial tissue.

Besides making a one-stage procedure feasible, the invention may result in one or more other advantages. In the case of a vascular prosthesis, fewer endothelial cells will be washed away from a luminal surface that includes recesses. As a result, the prosthesis maintains a high population of endothelial cells and can grow a confluent layer of cells in a short time. The prosthesis may also support in situ growth. If cell recesses are formed on substantially less than the full luminal surface of the prosthesis and if the seeding procedure deposits seeded cells onto the regions with recesses, fewer harvested cells are needed to seed the prosthesis. The harvested cells can be concentrated into cell-rich regions on the luminal surface supportive of rapid cell growth. The surface regions with cell recesses can be contiguous or interconnected by cell recess-containing paths to support formation of an endothelialized luminal surface. The patient benefits from the presence and health of the endothelial cells.

Moreover, various embodiments of the invention take advantage of physical properties of ePTFE, a material that has a proven track record in implantable medical devices. This material is biocompatible, and handles and sutures well. The techniques described herein for forming recesses do not adversely affect the favorable features of ePTFE. In addition, because the processing does not affect the overall integrity of the ePTFE, the material remains clinically usable even if no seeding is performed. The “fish-scale” pattern may also offer an equivalent or better hemocompatibility than conventional ePTFE. Further, processing of ePTFE as described herein may change the permeability of the ePTFE, which may be advantageous in some applications.

Various embodiments of the invention have been described. The invention is not limited to the particular embodiments described above. In particular, the invention is not limited to vascular prostheses that include ePTFE. Although many implantable devices use ePTFE, other biocompatible materials also may used to form vascular prostheses, and may be processed as described above to create recesses sized to receive endothelial cells.

In addition, ePTFE may be included in implantable medical devices other than vascular prostheses, some of which are mentioned above. For some implantable medical devices, the device may be seeded with developed or precursor endothelial cells, but the invention is not limited to seeding with endothelial cells. Some implantable medical devices may be seeded with other kinds of human, non-human or genetically engineered cells. For some implantable medical devices, no seeding is necessary at all.

Moreover, the invention is not limited to use of any particular tool or apparatus. There are many techniques for creating recesses, and the invention is not limited to the particular illustrative techniques described herein. The recesses need not be arranged in a “fish-scale” pattern. These and other embodiments are within the scope of the following claims.

What is claimed is:

1. A method for preparing a vascular prosthesis, comprising:
applying a frictional force to a luminal surface of said vascular prosthesis so as to create a substantially fibril-free surface of lifted nodes that define a plurality of recesses.

2. A method as in claim 1 wherein said vascular prosthesis comprises expanded polytetrafluoroethylene.

3. A method as in claim 1 wherein said vascular prosthesis has a generally tube-shaped structure having an axis, and wherein the frictional force is applied along an axial direction.

4. A method as in claim 3 wherein the frictional force is applied by a tool.

5. A method as in claim 4 wherein said tool is a wheel brush comprising bristles.

6. A method as in claim 5 wherein applying the frictional force comprises rubbing said luminal surface with said bristles.
